(19) United States
(12) Patent Application Publication
(10) Pub. No.: US 2015/0344382 A1
(43) Pub. Date: Dec. 3, 2015

(54) SYSTEMS AND METHODS FOR HYDROGEN SELF-SUFFICIENT PRODUCTION OF RENEWABLE HYDROCARBONS

(71) Applicant: UOP LLC, Des Plaines, IL (US)

(72) Inventors: Donald A. Eizenga, Elk Grove Village, IL (US); Daniel L. Ellig, Arlington Heights, IL (US); Tom N. Kalnes, LaGrange, IL (US)

(73) Assignee: UOP LLC, Des Plaines, IL (US)

(21) Appl. No.: 14/292,527

(22) Filed: May 30, 2014

(51) Int. Cl. C07C 1/207

(52) U.S. Cl. CPC C07C 1/2078

(57) ABSTRACT

Methods and systems for hydrogen self-sufficient production of hydrocarbons from a renewable feedstock are provided. An exemplary method includes providing a renewable feedstock; contacting the renewable feedstock and hydrogen from a hydrogen stream with one or more catalysts to generate an effluent comprising n-paraffins and by-product hydrocarbons having 9 or fewer carbon atoms; separating the by-product hydrocarbons from the effluent to generate a hydrocarbon by-product stream; and feeding the hydrocarbon by-product stream to a hydrogen plant to generate the hydrogen stream. In this exemplary embodiment, the by-product hydrocarbons constitute the entire feed and fuel of the hydrogen plant, and wherein no hydrogen is added from an external source.

### TECHNICAL FIELD

The technical field generally relates to systems and methods for producing hydrocarbons, and more particularly relates to systems and methods for hydrogen self-sufficient production of hydrocarbons from renewable feedstocks.

### BACKGROUND

Given the worldwide demand for hydrocarbons such as transportation fuel and paraffins, there is increasing interest in use of feedstocks other than petroleum crude oil for hydroprocessing. One category of alternative feedstocks has been termed renewable feedstocks. Examples of renewable feedstocks include plant oils such as corn, rapeseed canola, soybean and algal oils, animal fats and oils such as tallow, fish oils and various waste streams such as yellow and brown greases and sewage sludge. Processing renewable feedstocks involves hydrogenation, decarboxylation, decarbonylation, and/or hydrodeoxygenation and optionally hydroisomerization and cracking (or selective cracking) in one or more steps. Processing renewable feedstocks requires contacting the feedstock with hydrogen under catalytic hydroprocessing conditions. Normally, desired product specifications and yields are determined and reaction and fractionation conditions are set to optimize production of the desired products and minimize production of less economically valuable by-products.

In some cases it may be advantageous to have a hydroprocessing facility located near the source of a renewable feedstock, which is often remote from other infrastructure that is often necessary to provide a source of hydrogen or feed and/or fuel for a hydrogen producing process or system. For instance, if readily available, natural gas would normally be used as feed and fuel in the production of hydrogen that would then be used in the hydroprocessing of the renewable feedstock. However, in remote locations, a low cost, reliable source of natural gas may not be available. Accordingly, it is desirable to provide systems and methods for producing hydrocarbons from renewable feedstocks that are hydrogen self-sufficient. Furthermore, other desirable features and characteristics of the present invention will become apparent from the subsequent detailed description and the appended claims, taken in conjunction with the accompanying drawings and this background.

### BRIEF SUMMARY

Methods for hydrogen self-sufficient production of hydrocarbons from a renewable feedstock are provided herein. In accordance with an exemplary embodiment, a method includes: providing a renewable feedstock; contacting the renewable feedstock and hydrogen from a hydrogen stream with one or more catalysts to generate an effluent comprising n-paraffins and by-product hydrocarbons having 9 or fewer carbon atoms; separating the by-product hydrocarbons from the effluent to generate a hydrocarbon by-product stream; and feeding the hydrocarbon by-product stream as feed and fuel for a hydrogen plant to generate the hydrogen stream. In this embodiment, the by-product hydrocarbons constitute the entire feed and fuel of the hydrogen plant, and no hydrogen is added from an external source.

Also provided herein are systems for hydrogen self-sufficient production of hydrocarbons from a renewable feedstock. In one exemplary embodiment, a system includes a reaction zone configured to contain a hydrogenation and deoxygenation catalyst. The reaction zone is configured to receive and contact a renewable feedstock and hydrogen gas with the hydrogenation and deoxygenation catalyst under reaction conditions effective to generate n-paraffins and hydrocarbon by-products having 9 or fewer carbon atoms. Further, the reaction zone is configured to contain an isomerization and hydrocracking catalyst. The reaction zone is configured to contact the n-paraffins from the hydrogenation and deoxygenation catalyst and hydrogen with the isomerization and hydrocracking catalyst under reaction conditions effective to generate an effluent comprising hydrocarbons with a boiling point in the diesel boiling point range and hydrocarbon by-products having 9 or fewer carbon atoms. This exemplary system further includes a separation zone configured to receive an effluent from the reaction zone and fractionate the effluent into a first product stream comprising a diesel component with hydrocarbons with a boiling point in the diesel boiling point range and a hydrocarbon by-product stream comprising the by-product hydrocarbons; and a hydrogen plant configured to receive the hydrocarbon by-product stream as feed and fuel for the generation of hydrogen. In this exemplary system, the hydrogen plant is further configured such that by-product hydrocarbons constitute the entire feed and fuel of the hydrogen plant, and wherein no hydrogen is added to the system from an external source.

In another exemplary system, the system includes a first reaction zone configured to contain a hydrogenation and deoxygenation catalyst. The first reaction zone is configured to receive and contact a renewable feedstock and hydrogen gas with the hydrogenation and deoxygenation catalyst under reaction conditions effective to generate n-paraffins and hydrocarbon by-products having 9 or fewer carbon atoms. The system also includes a first separation zone configured to receive an effluent from the first reaction zone and fractionate the effluent into a first product stream comprising n-paraffins with 10 to 13 carbon atoms, a second product stream comprising hydrocarbons with 14 or more carbon atoms, and a first hydrocarbon by-product stream comprising by-product hydrocarbons having 9 or fewer carbon atoms. Further, the system includes a hydrogen plant configured to receive the first hydrocarbon by-product stream as feed and fuel for the generation of hydrogen. In this exemplary system, the hydrogen plant is further configured such that the hydrogen plant does not receive any feed or fuel from an external source, and wherein no hydrogen is added to the system from an external source.

### DETAILED DESCRIPTION OF THE DRAWINGS

The various embodiments will hereinafter be described in conjunction with the following drawing figures, wherein like numerals denote like elements, and wherein:

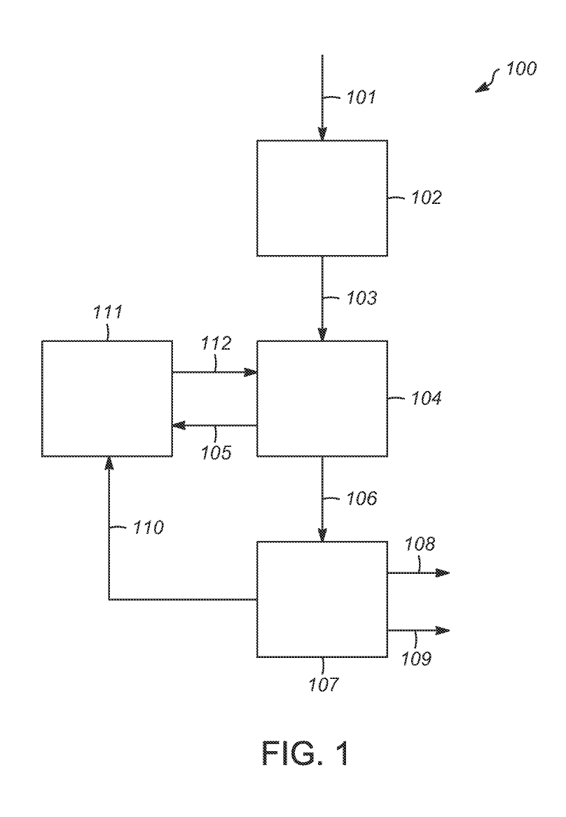

FIG. 1 is an illustration of the process flow of a hydrogen self-sufficient system for production of a diesel fuel component and an aviation fuel component from a renewable feedstock.

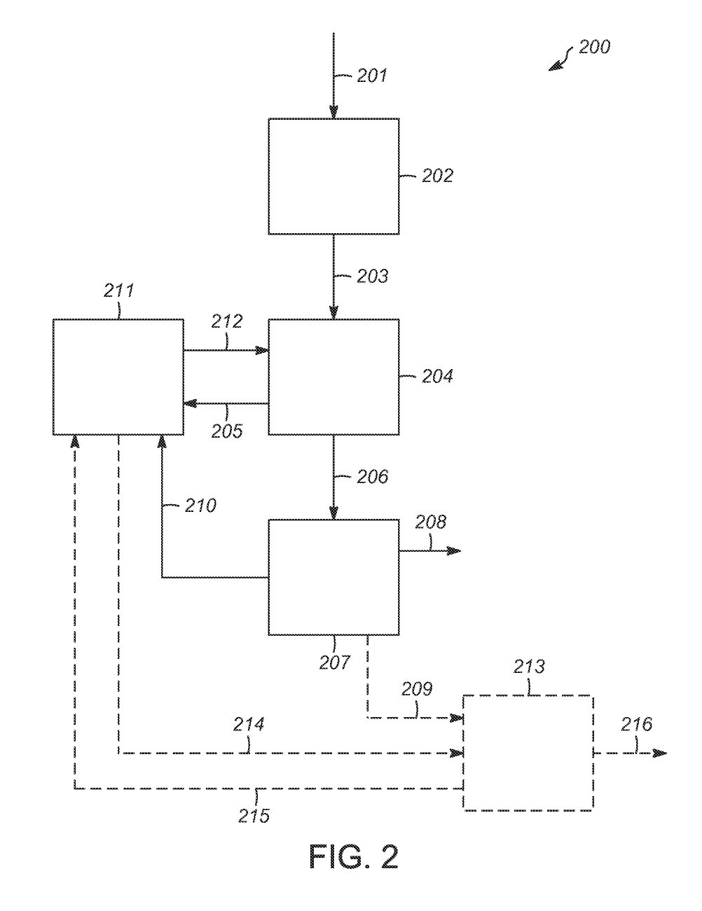

FIG. 2 is an illustration of the process flow of a hydrogen self-sufficient system for production of n-paraffins and optionally a diesel fuel component from a renewable feedstock.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

As provided above, systems and methods are described herein for the hydrogen self-sufficient production of hydrocarbons from renewable feedstocks. Production of hydrocarbons from renewable feedstocks involves hydrogenation, decarboxylation, decarbonylation, and/or hydrodeoxygenation and optionally hydroisomerization and hydrocracking (or selective hydrocracking), in one or more steps. These processes result in production of one or more desired hydrocarbons (such as paraffins with a desired number of carbons and/or one or more transportation fuels) and one or more hydrocarbon by-products. As used herein, hydrocarbons having 9 or fewer carbon atoms are typically considered hydrocarbon by-products. Specific renewable hydrocarbon by-products may include, but are not limited to, naphtha, liquefied renewable gas (also known herein as LPG), and hydrocarbon gases having 3 or fewer carbon atoms. These hydrocarbon by-products are suitable for use as fuel and feedstock for the production of hydrogen by steam reforming in a hydrogen plant. The resulting hydrogen may then be utilized as a co-reactant with the renewable feedstock.

Typically, the conditions for production of hydrocarbons from renewable feedstocks are controlled such that generation of desired hydrocarbons is maximized, and generation of hydrocarbon by-products having 9 or fewer carbon atoms is minimized. However, operating under these conditions does not always provide sufficient quantities of hydrocarbon by-products so as to allow for hydrogen self-sufficiency. That is, operating under typical conditions may not provide sufficient quantities of hydrocarbon by-products for generation of all necessary hydrogen via a hydrogen plant. Thus, systems and methods utilizing typical operating conditions are not hydrogen self-sufficient, but rather require supplementation of hydrogen with an external source of fuel and/or feed (typically a fossil fuel such as natural gas) for the hydrogen plant, or addition of hydrogen from an external source, to supply all necessary hydrogen for the production of hydrocarbons from a renewable feedstock.

In the systems and methods described herein, the conditions for production of hydrocarbons are set such that generation of the desired hydrocarbons is reduced relative to the maximum possible for a given feedstock, and production of hydrocarbon by-products is increased. This increased generation of hydrocarbon by-products may provide sufficient fuel and feedstock for hydrogen generation so that no external source of hydrogen, or external source of fuel and/or feedstock for the generation of hydrogen, is needed. Thus, the systems and methods provided herein can operate without input of any fossil fuel as feed and/or fuel for hydrogen generation.

In some embodiments, the systems and methods provided herein are useful for processing a renewable feedstock to generate one or both of a diesel fuel component and an aviation fuel component. In some alternate embodiments, the systems and methods provided herein are useful for processing a renewable feedstock to generate an n-paraffin containing effluent. In either case, conditions for production of hydrocarbons are set such that generation of the desired hydrocarbons is reduced relative to the maximum possible for a given feedstock, as described above. Conventionally, operating conditions for the production of hydrocarbons from renewable feedstocks are set so as to result in the maximum or about the maximum amount of the desired hi-value hydrocarbon product possible. Thus, under normal circumstances, operating conditions are set so as to maximize revenue that can be realized from a given feedstock. This is achieved by minimizing the amount of hydrocarbon by-products that are produced. Embodiments described herein differ in that operating conditions are selected to reduce production of conventionally desirable hydrocarbon products, and enhance production of conventionally less desirable hydrocarbon by-products. The operating conditions used herein are set so as to ensure that sufficient hydrogen can be generated from the hydrocarbon by-products so that production of renewable hydrocarbons can be hydrogen self-sufficient. As indicated above, this means that production of renewable hydrocarbons can be accomplished without the input of any fossil fuel as feed and/or fuel for hydrogen generation.

As used herein, the term renewable feedstock is meant to include feedstocks other than those obtained directly from petroleum crude oil. Another term that has been used to describe at least a portion of this class of feedstocks is biorenewable feedstocks. Renewable feedstocks include any of those which comprise glycerides, fatty acid alkyl esters (FAAE), and free fatty acids (FFA). Examples of these feedstocks include, but are not limited to, canola oil, corn oil, soy oils, rapeseed oil, soybean oil, colza oil, tall oil, sunflower oil, hempseed oil, olive oil, linseed oil, coconut oil, castor oil, peanut oil, palm kernel oil, mustard oil, cottonseed oil, tallow, yellow and brown greases, lard, train oil, fats in milk, fish oil, algal oil, sewage sludge, cuphea oil, camelina oil, jatropha oil, curcas oil, babassu oil, palm oil, crambe oil, fatty acid methyl esters, lard, kernel oil, used cooking oil, animal fats, and the like. In some particular embodiments, the renewable feedstock is palm oil or coconut oil.

The glycerides, FAAES and FFAs of typical vegetable or animal fats contain aliphatic hydrocarbon chains in their structure which have about 8 to about 24 carbon atoms with many of the oils containing high concentrations of fatty acids with 16 and 18 carbon atoms. The aliphatic carbon chains in the glycerides, FFAs, or FAAEs can be saturated or mono-, di- or poly-unsaturated. Most of the glycerides in the renewable feed stocks will be triglycerides, but some of the glycerides in the renewable feedstock may be monoglycerides or diglycerides. The monoglycerides and diglycerides can be processed along with the triglycerides.

In some embodiments, renewable feedstocks may be mixed or co-fed with petroleum derived hydrocarbons. Other feedstock components which may be used, especially as a co-feed component in combination with the above listed renewable feedstocks, include spent motor oils and industrial lubricants, used paraffin waxes, liquids derived from gasification of coal, biomass, or natural gas followed by a downstream liquefaction step such as Fischer-Tropsch technology; liquids derived from depolymerization, thermal or chemical, of waste plastics such as polypropylene, high density polyethylene, and low density polyethylene; and other synthetic oils generated as byproducts from petrochemical and chemical processes. Mixtures of the above feedstocks may also be used as co-feed components. One advantage of using a co-feed component is transformation of what has been considered to be a waste product from a petroleum based process into a valuable co-feed component to the current process.

There are a number of examples in the art disclosing the production of hydrocarbons from plant oils. For example, U.S. Pat. No. 4,300,009 discloses the use of crystalline aluminosilicate zeolites to convert plant oils such as corn oil to hydrocarbons such as gasoline and chemicals such as para-xylene. U.S. Pat. No. 4,992,605 discloses the production of hydrocarbon products in the diesel boiling range by hydroprocessing vegetable oils such as canola or sunflower oil. Finally, U.S. Pat. No. 7,232,935 discloses a process for treating a hydrocarbon component of biological origin by hydrodeoxygenation followed by isomerization.

Methods and systems for the generation of transportation fuels will first be addressed, however, it should be understood that several of the steps and components described below (optional pretreatment steps, reactors, catalysts, separation of effluent components via fractionation, etc.) may also be used in methods and systems for the generation of n-paraffins. Thus, while the following description is in the context of generation of transportation fuels, it should be understood that the steps and components that follow are not limited as such.

In some embodiments, methods of generating transportation fuels, such as a diesel and aviation fuels, comprise an optional pretreatment step and one or more steps to hydrogenate, deoxygenate, hydroisomerize and optionally hydrocrack the renewable feedstock, to generate both a diesel fuel component and an aviation fuel component. In these embodiments, the diesel component and the aviation component may be suitable as fuels, used as components of blending pools, or may have one or more additives incorporated before being used as fuels.

The diesel component comprises hydrocarbons having a boiling point in the diesel boiling point range and may be used directly as a fuel, may be blended with other components before being used as diesel fuel, or may receive additives before being used as a diesel fuel. As used herein, the diesel fuel boiling point range is about 120° C. to about 370° C. The aviation component comprises hydrocarbons having a boiling point in the aviation fuel boiling point range, which includes the jet fuel range, and may be used directly as aviation fuel or may be used as a blending component to meet the specifications for a specific type of aviation fuel, or may receive additives before being used as an aviation fuel. As used herein, the aviation fuel boiling point range is about 120° C. to about 285° C. Depending upon the application, various additives may be combined with the aviation component or the diesel component generated in order to meet required specifications for different specific fuels. In particular, the aviation fuel composition generated herein complies with, is a blending component for, or may be combined with one or more additives to meet ASTM D 7566 Standard Specification for Aviation Turbine Fuel Containing Synthesized Hydrocarbons. The aviation fuel is generally termed “jet fuel” herein and the term “jet fuel” is meant to encompass aviation fuel meeting the specifications above as well as to encompass aviation fuel used as a blending component of an aviation fuel meeting the specifications above. Additives may be added to the jet fuel in order to meet particular specifications.

Systems and methods of the prior art typically start with desired specifications and relative yields of the diesel and aviation components, and operating conditions of an isomerization and hydrocracking zone are optimized to meet the desired specifications and relative yields while producing as little hydrocarbon by-product as possible. As described above, systems and methods provided herein differ in that the operating conditions of an isomerization and hydrocracking zone are not set to yield the maximum or about the maximum possible production of diesel and aviation components. Instead, conditions are set so as to increase production of conventionally less desirable hydrocarbon by-products, such as naphtha, LPG, and hydrocarbons with 3 or fewer carbon atoms.

The control of the process allows for an operator to select the specific product composition and the amount of hydrocarbon by-product that is produced. Specifically, the operating conditions of the isomerization and hydrocracking (or selective hydrocracking) zone, described below, are set so that the effluent of the zone comprises hydrocarbons necessary for the desired product composition, as well as an increased amount of hydrocarbon by-products having 9 or fewer carbon atoms, relative to conventional operating conditions for production of the desired product composition. The operating conditions of a fractionation zone, also described below, are determined so that the hydrocarbons produced in the isomerization and hydrocracking zone are separated into at least two product streams: a first product stream comprising hydrocarbons with a boiling point in the diesel fuel boiling range and meeting specifications selected for a diesel component and a second product stream comprising hydrocarbon by-products having 9 or fewer carbon atoms. In some embodiments, a third product stream may optionally be separated which comprises hydrocarbons with a boiling point in the aviation fuel boiling range and meeting specifications selected for an aviation component. The second product stream comprising hydrocarbon by-products is directed to a hydrogen plant, and used as feed and fuel for the generation of hydrogen, which is directed back to the isomerization and hydrocracking zone as necessary. Conventional hydrogen plants that can operate with the hydrocarbon by-product as feed and fuel for the generation of hydrogen (such as a standard steam reformer) may be employed.

In embodiments, renewable feedstocks may be used that contain a variety of impurities. For example, tall oil is a by-product of the wood processing industry and tall oil contains esters and rosin acids in addition to FFAs. Rosin acids are cyclic carboxylic acids. The renewable feedstocks may also contain contaminants such as alkali metals, e.g. sodium and potassium, phosphorous as well as solids, water and detergents. An optional first step is to reduce or remove contaminants from the feedstock before processing. One possible pretreatment step involves contacting the renewable feedstock with an ion-exchange resin in a pretreatment zone at pretreatment conditions. The ion-exchange resin is an acidic ion exchange resin such as Amberlyst®-15 and can be used as a bed in a reactor through which the feedstock is flowed through, either upflow or downflow. Another technique includes contacting the renewable feedstock with a bleaching earth, such as bentonite clay, in a pretreatment zone.

Another possible technique for reducing or removing contaminants is a mild acid wash. This is carried out by contacting the renewable feedstock with an acid such as sulfuric, nitric, phosphoric, or hydrochloric in a reactor. The acid and renewable feedstock can be contacted either in a batch or continuous process. Contacting is done with a dilute acid solution usually at ambient temperature and atmospheric pressure. If the contacting is done in a continuous manner, it is usually done in a counter current manner. Yet another possible technique for reducing or removing metal contaminants from the renewable feedstock is through the use of conventional guard beds. These can include alumina guard beds either with or without demetallation catalysts such as nickel or cobalt. Filtration and solvent extraction techniques are other choices which may be employed. Hydroprocessing such as that described in U.S. Pat. No. 7,638,040 is another pretreatment technique which may be employed.

Further, any other conventional technique may be used to reduce or remove contaminants from a renewable feedstock as desired. For example, in some embodiments a renewable feedstock, such as a palm oil derived feedstock, may be fractionated to reduce or remove impurities.

With the specifications of the products being determined, the relative yields of the products being determined, and the operating conditions determined and set so as to increase production of light hydrocarbon by-product, the feedstock is flowed to a reaction zone comprising one or more catalyst beds in one or more reactors. The term feedstock is meant to include feedstocks that have not been treated to remove contaminants as well as those feedstocks purified in a pretreatment zone or oil processing facility. In the reaction zone, the feedstock is contacted with a hydrogenation or hydrotreating catalyst in the presence of hydrogen at hydrogenation conditions to hydrogenate the olefinic or unsaturated portions of the aliphatic hydrocarbon chains. Examples of suitable hydrogenation or hydrotreating catalysts include, but are not limited to, nickel or nickel/molybdenum dispersed on a high surface area support. Other hydrogenation catalysts include one or more noble metal catalytic elements dispersed on a high surface area support. Non-limiting examples of noble metals include Pt and/or Pd dispersed on gamma-aluminas. Hydrogenation conditions include an inlet temperature of about 100° C. to about 400° C., such as about 250° C. to about 400° C., such as about 250° C. to about 300° C., and a pressure of about 690 kPa absolute (100 psia) to about 10343 kPa absolute (1500 psia), such as about 1379 kPa absolute (200 psia) to about 5516 kPa absolute (800 psia). Other conventional operating conditions for the hydrogenation zone may be employed. In some specific embodiments, hydrogenation conditions for feedstocks predominantly comprising plant based oils may include a pressure of about 1379 kPa absolute (200 psia) to about 4826 kPa absolute (700 psia). In other specific embodiments, hydrogenation conditions for feedstocks predominantly comprising animal fats or waste oils may include a pressure of about 3447 kPa absolute (500 psia) to about 5516 kPa absolute (800 psia). In some embodiments, the reactor outlet temperature is about 400° C. to about 500° C.

The hydrogenation and hydrotreating catalysts enumerated above are also capable of catalyzing decarboxylation, decarbonylation, and/or hydrodeoxygenation of the feedstock to remove oxygen. Decarboxylation, decarbonylation, and hydrodeoxygenation are herein collectively referred to as deoxygenation reactions. Deoxygenation conditions may include a relatively low pressure of about 1724 kPa absolute (250 psia) to about 10.342 kPa absolute (1500 psia), with embodiments in the range of 3447 kPa (500 psia) to about 6895 kPa (1000 psia) or below 4826 kPaa (700 psia); a temperature of about 200° C. to about 460° C. with embodiments in the range of about 271° C. to about 382° C.; and a liquid hourly space velocity of about 0.25 to about 4 hr⁻¹ with embodiments in the range of about 1 to about 4 hr⁻¹. Because hydrogenation is an exothermic reaction, the temperature of the catalyst bed increases as the feedstock flows through the reactor and decarboxylation, decarbonylation, and hydrodeoxygenation occur. Although the hydrogenation reaction is exothermic, some feedstocks may be highly saturated and not generate enough heat internally. Therefore, some embodiments may require external heat input.

The reaction product from the hydrogenation and deoxygenation reactions comprises both a liquid fraction and a gaseous fraction. The liquid fraction comprises a hydrocarbon fraction comprising n-paraffins and having a large concentration of paraffins in the 10 to 18 carbon number range. Different feedstocks will result in reaction products with different distributions of paraffins. In some embodiments, a portion of the liquid hydrocarbon fraction may be recycled through the deoxygenation reactor for heat management. Although the liquid hydrocarbon fraction is useful as a diesel fuel or diesel fuel blending component, additional fuels, such as aviation fuels or aviation fuel blending components which typically have a concentration of paraffins in the range of about 9 to about 15 carbon atoms, may be produced with additional processing, i.e., isomerization and cracking. Also, because the hydrocarbon fraction comprises essentially all n-paraffins, it will have poor cold flow properties. Many diesel and aviation fuels and blending components must have better cold flow properties and so in some embodiments a portion of the reaction product comprising n-paraffins in the 14 to 18 carbon number range is further reacted under isomerization conditions to isomerize at least a portion of the n-paraffins to branched paraffins.

The gaseous portion of the reaction product from the hydrogenation and deoxygenation zone comprises hydrogen, carbon dioxide, carbon monoxide, water vapor, propane nitrogen or nitrogen compounds and perhaps sulfur components such as hydrogen sulfide. The effluent from the deoxygenation zone may be sent to a hot high pressure hydrogen stripper. One purpose of a hot high pressure hydrogen stripper is to selectively separate at least a portion of the gaseous portion of the effluent from the liquid portion of the effluent. To facilitate hydrogen self-sufficiency, the separated hydrogen is recycled to the first reaction zone containing the deoxygenation reactor. Also, failure to remove the water, carbon monoxide, and carbon dioxide from the effluent may result in poor catalyst performance in the isomerization zone. Water, carbon monoxide, carbon dioxide, any ammonia or hydrogen sulfide are selectively stripped in the hot high pressure hydrogen stripper using hydrogen. The hydrogen used for the stripping may be dry and free of carbon oxides. The temperature may be controlled in a limited range to achieve the desired separation and the pressure may be maintained at approximately the same pressure as the two reaction zones to minimize both investment and operating costs. The hot high pressure hydrogen stripper may be operated at conditions ranging from a pressure of about 689 kPa absolute (100 psia) to about 13,790 kPa absolute (2000 psia), and a temperature of about 40° C. to about 350° C. In another embodiment the hot high pressure hydrogen stripper may be operated at conditions ranging from a pressure of about 1379 kPa absolute (200 psia) to about 4826 kPa absolute (700 psia), or about 2413 kPa absolute (350 psia) to about 4882 kPa absolute (650 psia), and a temperature of about 50° C. to about 350° C.

In some embodiments, the hot high pressure hydrogen stripper is operated at essentially the same pressure as the reaction zone. By “essentially” it is meant that the operating pressure of the hot high pressure hydrogen stripper is within about 1034 kPa absolute (150 psia) of the operating pressure of the reaction zone. For example, in one embodiment the operating pressure of the hot high pressure hydrogen stripper separation zone is less than that of the reaction zone, but is within 1034 kPa absolute (150 psia).

The effluent enters the hot high pressure stripper and at least a portion of the gaseous components are carried with the hydrogen stripping gas and separated into an overhead stream. The remainder of the deoxygenation zone effluent stream is removed as hot high pressure hydrogen stripper bottoms and contains the liquid hydrocarbon fraction having components such as normal hydrocarbons having from about 8 to 24 carbon atoms. At least a portion of this liquid hydrocarbon fraction in hot high pressure hydrogen stripper bottoms may be used as a hydrocarbon recycle as described in U.S. Pat. No. 7,982,078.

As described above, although the hydrocarbons in the liquid portion of the reaction product may be useful as a diesel fuel, or a diesel fuel blending component, these hydrocarbons are essentially all n-paraffins and will have poor cold flow properties. To improve the cold flow properties of the liquid hydrocarbon fraction, the reaction product can be contacted with an isomerization catalyst under isomerization conditions in an isomerization and hydrocracking zone to at least partially isomerize the n-paraffins to isoparaffins. Additionally, the conditions in the isomerization and hydrocracking zone may be increased in severity so as to produce an increased amount of light hydrocarbon by-products. In some embodiments, the conditions of the isomerization and hydrocracking zone may be set such that the amount of hydrocarbon by-products produced is about 5 wt % to about 40 wt %, such as about 10 wt % to about 40 wt %, such as about 15 wt % to about 40 wt %, such as about 20 wt % to about 40 wt %, of the fresh feed. In some embodiments, the amount of hydrocarbon by-products produced is about 10 wt % to about 30 wt % of the fresh feed.

Conventional catalysts and conditions for isomerization may be employed. Isomerization can be carried out in a separate bed of the same reaction zone, i.e. same reactor, described above or the isomerization can be carried out in a separate reactor. The product of the deoxygenation reaction zone is contacted with an isomerization catalyst in the presence of hydrogen at isomerization conditions to isomerize the normal paraffins to branched paraffins. In some embodiments, only minimal branching is required, enough to overcome cold-flow problems of the normal paraffins. In other embodiments, a greater amount of isomerization is desired. The predominate isomerization product is generally a mono-branched hydrocarbon. Along with the isomerization, some hydrocracking of the hydrocarbons will occur. The more severe the conditions of the isomerization zone, the greater the amount of hydrocracking of the hydrocarbons. The hydrocracking occurring in the isomerization zone results in a wider distribution of hydrocarbons than resulted from the deoxygenation zone and increased levels of hydrocracking produces higher yields of hydrocarbons in the aviation fuel boiling range. Additionally, the conditions in the isomerization and hydrocracking zone may be increased in severity to produce an increased amount of light hydrocarbon by-products.

The isomerization of the paraffinic hydrocarbons can be accomplished in any conventional manner or by using any suitable conventional catalyst. Suitable catalysts comprise a metal of Group VIII (IUPAC 8-10) of the Periodic Table and a support material. Suitable Group VIII metals include platinum and palladium, each of which may be used alone or in combination. The support material may be amorphous or crystalline. Suitable support materials include aluminas, amorphous aluminas, amorphous silica-aluminas, ferrierite, ALPO-31, SAPO-11, SAPO-31, SAPO-37, SAPO-41, SM-3, MgAPSO-31, FU-9, NU-10, NU-23, ZSM-12, ZSM-22, ZSM-23, ZSM-35, ZSM-48, ZSM-50, ZSM-57, MeAPO-11, MeAPO-31, MeAPO-41, MgAPSO-11, MgAPSO-31, MgAPSO-41, MgAPSO-46, ELAPO-11, ELAPO-31, ELAPO-41, ELAPSO-11, ELAPSO-31, ELAPSO-41, laumontite, cancrinite, offretite, hydrogen form of stillbite, magnesium or calcium form of mordenite, and magnesium or calcium form of partheite, each of which may be used alone or in combination. ALPO-31 is described in U.S. Pat. No. 4,310,440. SAPO-11, SAPO-31, SAPO-37, and SAPO-41 are described in U.S. Pat. No. 4,440,871. SM-3 is described in U.S. Pat. Nos. 4,943,424; 5,087,347; 5,158,665; and 5,208,005. MgAPSO is a MeAPSO, which is an acronym for a metal aluminumsilicophosphate molecular sieve, where the metal Me is magnesium (Mg). Suitable MgAPSO-31 catalysts include MgAPSO-31. MeAPSOs are described in U.S. Pat. No. 4,793,984, and MgAPSOs are described in U.S. Pat. No. 4,758,419. MgAPSO-31 is a preferred MgAPSO, where 31 means a MgAPSO having structure type 31. Many natural zeolites, such as ferrierite, that have an initially reduced pore size can be converted to forms suitable for olefin skeletal isomerization by removing associated alkali metal or alkaline earth metal by ammonium ion exchange and calcination to produce the substantially hydrogen form, as taught in U.S. Pat. Nos. 4,795,623 and 4,924,027. Further catalysts and conditions for skeletal isomerization are disclosed in U.S. Pat. Nos. 5,510,306, 5,082,956, and 5,741,759.

The isomerization catalyst may also comprise a modifier selected from the group consisting of lanthanum, cerium, praseodymium, neodymium, samarium, gadolinium, terbium, and mixtures thereof, as described in U.S. Pat. Nos. 5,716,897 and 5,851,949. Other suitable support materials include ZSM-22, ZSM-23, and ZSM-35, which are described for use in dewaxing in U.S. Pat. No. 5,246,566.

U.S. Pat. Nos. 5,444,032 and 5,608,968 teach a suitable bifunctional catalyst which is constituted by an amorphous silica-alumina gel and one or more metals belonging to Group VIIIA, and is effective in the hydroisomerization of long-chain normal paraffins containing more than 15 carbon atoms. U.S. Pat. Nos. 5,981,419 and 5,908,134 teach a suitable bifunctional catalyst which comprises: (a) a porous crystalline material isostructural with beta-zeolite selected from boro-silicate (BOR-B) and boro-alumino-silicate (Al-BOR-B) in which the molar SiO₂:Al₂O₃ ratio is higher than 300:1; (b) one or more metal(s) belonging to Group VIIIA, selected from platinum and palladium, in an amount comprised within the range of from 0.05 to 5% by weight. Other suitable catalysts are known in the art.

In general, isomerization conditions include a temperature of about 150° C. to about 450° C. and a pressure of about 1724 kPa absolute (250 psia) to about 5516 kPa absolute (800 psia), such as about 150° C. to about 390° C. and a pressure of about 1724 kPa absolute (250 psia) to about 4726 kPa absolute (700 psia). In another embodiment the isomerization conditions include a temperature of about 350° C. to about 390° C. and a pressure of about 3102 kPa absolute (450 psia) to about 3792 kPa absolute (550 psia). Other conventional operating conditions for the isomerization zone may be employed, and the specific operating conditions used depend on the desired product specifications and amount of hydrocarbon by-product necessary to achieve hydrogen self-sufficiency.

The catalysts suitable for the isomerization of the paraffinic hydrocarbons and conditions of the isomerization zone also operate to cause some hydrocracking of the hydrocarbons. Therefore, although a main product of the hydrogenation, deoxygenation, and isomerization steps is a paraffinic hydrocarbon fraction suitable for use as diesel fuel or as a blending component for diesel fuel, a second paraffinic hydrocarbon suitable for use as an aviation fuel, or as a component for aviation fuel is also generated. As illustrative of this concept, a concentration of paraffins formed from renewable feedstocks typically has about 15 to 18 carbon atoms, but additional paraffins may be formed to provide a range of from about 3 to about 24 carbon atoms. A portion of the normal paraffins are isomerized to branched paraffins but the carbon number range of paraffins does not alter with isomerization alone. However, some hydrocracking will occur concurrently with the isomerization, generating paraffins having boiling points from about 150° C. to about 250° C., which is lower than that of the majority of C15 to C18 paraffins produced in the deoxygenation reaction zone. The about 150° C. to about 250° C. boiling point range meets many aviation fuel specifications and can therefore be separated from the other boiling point ranges after the isomerization zone in order to produce an aviation fuel. This will lower the overall yield of diesel fuel but allows the production of two fuel products: a diesel fuel and an aviation fuel. The process severity in the isomerization zone controls the potential yield of product for aviation fuel, the amount of light products that are not useful for diesel fuel or aviation fuel, and the isomerized/normal ratio of both aviation and diesel range fuel.

When feed and fuel for hydrogen production is easily obtained, hydrocracking is controlled through catalyst choice and reaction conditions in an attempt to restrict the degree of hydrocracking that occurs so as to maximize production of desired hydrocarbons and minimize production of hydrocarbon by-products. However, in the systems and methods described herein, the choice of catalyst and control of the process conditions in the isomerization zone is such that production of hydrocarbon by-products having 9 or fewer carbon atoms that are not useful for either diesel fuel or aviation fuel applications is encouraged to the point that hydrogen self-sufficiency can be achieved.

Fuel specifications are typically not based upon carbon number ranges. Instead, the specifications for different types of fuels are often expressed through acceptable ranges of chemical and physical requirements of the fuel with the written specification of various types being periodically revised. Often a distillation range from 10 percent recovered to a final boiling point is used as a key parameter defining different types of fuels. The distillations ranges are typically measured by ASTM Test Method D 86 or D2887. Therefore, blending of different components in order to meet the specification is quite common. While the aviation fuel product of the present invention may meet aviation fuel specifications, it is expected that some blending of the product with other blending components may be required to meet the desired set of fuel specifications. In other words, one product of the systems and methods described herein is a composition which may be used with other components to form a fuel meeting at least one of the specifications for aviation fuel such as Jet A or Jet A-1. The desired aviation fuel product is a highly paraffinic distillate fuel component having a paraffin content of at least 75% by volume.

The catalysts of the subject systems and methods can be formulated using industry conventional techniques. Catalysts may be manufactured in the form of a cylindrical extrudate having a diameter of from about 0.8 to about 3.2 mm ( 1/32 in to about ⅛ in), or can be made in any other desired form such as a sphere or pellet. The extrudate may be in forms other than a cylinder such as the form of a well-known trilobe or other shape which has advantages in terms or reduced diffusional distance or pressure drop.

The stream obtained after all reactions have been carried out, the final effluent stream, is now processed through one or more separation steps to obtain at least two purified hydrocarbon product streams, one useful as a diesel fuel or diesel fuel blending component, and a second stream of hydrocarbon by-products having 9 or fewer carbon atoms (i.e., a hydrocarbon by-product stream). Optionally, a third purified hydrocarbon stream useful as aviation fuel or an aviation fuel blending component may also be obtained.

With the effluent stream of the isomerization and hydrocracking zone comprising both a liquid component and a gaseous component, various portions of which may be recycled, multiple separation steps may be employed. For example, hydrogen may be first separated in an isomerization effluent separator with the separated hydrogen being removed in an overhead stream. Suitable operating conditions of the isomerization effluent separator include, for example, a temperature of about 185° C. to about 275° C. and a pressure of about 3280 kPa absolute (480 psia) to about 4920 kPa absolute (720 psia). If there is a low concentration of carbon oxides, or the carbon oxides are removed, the hydrogen may be recycled back to the hot high pressure hydrogen stripper for use both as a rectification gas and to combine with the remainder as a bottoms stream.

The remainder of the isomerization effluent after the removal of hydrogen still has liquid and gaseous components and may be cooled, for instance by techniques such as air cooling or water cooling, and passed to a cold separator where the liquid component is separated from the gaseous component. Suitable operating conditions of a cold separator include, for example, a temperature of about 20° C. to about 60° C. and a pressure of about 3080 kPa absolute (450 psia) to about 4620 kPa absolute (670 psia). A water byproduct stream is also separated. In some embodiments, a portion of the liquid component, after cooling and separating from the gaseous component, may be recycled back to the isomerization zone to increase the degree of isomerization. Prior to entering a cold separator, the remainder of the isomerization and hydrocracking zone effluent may be combined with the hot high pressure hydrogen stripper overhead stream, and the resulting combined stream may be introduced into the cold separator.

The liquid component contains the hydrocarbons useful as diesel fuel and aviation fuel, as well as hydrocarbon by-products, such as naphtha and LPG. The separated liquid component is further purified in a product fractionation zone which separates lower boiling components and dissolved gases into an LPG and naphtha stream; an aviation range product; and a diesel range product. Suitable operating conditions of the product distillation zone include a temperature of from about 20° C. to about 200° C. at the overhead and a pressure from about 0 kPa (0 psia) to about 1379 kPa absolute (200 psia). The conditions of the distillation zone may be adjusted to control the relative amounts of hydrocarbon contained in the aviation range product stream and the diesel range product stream.

The light hydrocarbon by-product stream may be further separated in a debutanizer or depropanizer in order to separate the LPG, propane and light ends into an overhead stream, leaving the naphtha in a bottoms stream. Suitable operating conditions of this unit include a temperature of from about 20° C. to about 200° C. at the overhead and a pressure from about 0 kPa (0 psia) to about 2758 kPa absolute (400 psia). The hydrocarbons from the hydrocarbon by-product stream (including LPG and naphtha) are then used as feed and fuel for a hydrogen production facility, as described above.

In another embodiment, a single fraction column may be operated to provide four streams, with the hydrocarbons suitable for use in a diesel fuel removed from the bottom of the column, hydrocarbons suitable for use in an aviation fuel removed from a first side-cut, hydrocarbons in the naphtha range being removed in a second site-cut and the propane and light ends being removed in an overhead from the column. In yet another embodiment, a first fractionation column may separate the hydrocarbons useful in diesel and aviation fuels into a bottoms stream, and propane, light ends, and naphtha into an overhead stream. A second fractionation column may be used to separate the hydrocarbons suitable for use in a diesel fuel into a bottoms stream of the column and hydrocarbons suitable for use in an aviation fuel into an overhead stream of the column, while a third fractionation column may be employed to separate the naphtha range hydrocarbons from the propane and light ends. Also, dividing wall columns may be employed.

The operating conditions of the one or more fractionation columns may be used to control the amount of the hydrocarbons that are withdrawn in each of the streams as well as the composition of the hydrocarbon mixture withdrawn in each stream. Typical operating variables well known in the distillation art include column temperature, column pressure (vacuum to above atmospheric), reflux ratio, and the like. The result of changing column variables, however, is only to adjust the vapor temperature at the top of the distillation column. Therefore the distillation variables are adjusted with respect to a particular feedstock in order to achieve a temperature cut point to give a product that meets desired properties.

Optionally the process may employ a steam reforming zone as a hydrogen plant in order to provide hydrogen to the hydrogenation/deoxygenation zone and isomerization zone. The steam reforming process is a well known chemical process for producing hydrogen, and is the most common method of producing hydrogen or hydrogen and carbon oxide mixtures. A hydrocarbon and steam mixture is catalytically reacted at high temperature to form hydrogen, and the carbon oxides: carbon monoxide and carbon dioxide. Because the reforming reaction is strongly endothermic, heat must be supplied to the reactant mixture, such as by heating the tubes in a furnace or reformer. A specific type of steam reforming is autothermal reforming, also called catalytic partial oxidation. This process differs from catalytic steam reforming in that the heat is supplied by the partial internal combustion of the feedstock with oxygen or air, and not supplied from an external source. In general, the amount of reforming achieved depends on the temperature of the gas leaving the catalyst; exit temperatures in the range of about 700° C. to about 950° C. are typical for conventional hydrocarbon reforming Pressures may range up to about 4000 kPa absolute. Steam reforming catalysts are well known and conventional catalysts are suitable for use in the systems and methods described herein.

In an alternative embodiment, catalytic reforming may be employed instead of steam reforming In a typical catalytic reforming zone, the reactions include dehydrogenation, dehydrocyclization, isomerization, and hydrocracking. The dehydrogenation reactions typically will be the dehydroisomerization of alkylcyclopentanes to alkylcyclohexanes, the dehydrogenation of paraffins to olefins, the dehydrogenation of cyclohexanes to alkylcycloparaffins and the dehydrocyclization of acyclic paraffins and acyclic olefins to aromatics. The isomerization reactions included isomerization of n-paraffins to isoparaffins, the hydroisomerization of olefins to isoparaffins, and the isomerization of substituted aromatics. The hydrocracking reactions include the hydrocracking of paraffins. The aromatization of the n-paraffins to aromatics is generally considered to be highly desirable because of the high octane rating of the resulting aromatic product. In this application, the hydrogen generated by the reactions is also a highly desired product, for it is recycled to at least the deoxygenation zone. The hydrogen generated is recycled to any of the reaction zones, the hydrogenation/deoxygenation zone, the isomerization zone, and or the hydrocracking zone.

Turning to FIG. 1, in one exemplary embodiment the operator determines the amount of hydrocarbon by-products that will be necessary for hydrogen self-sufficiency of the system 100. The operator then determines the yield of each of an aviation component and a diesel component to be produced, while still ensuring hydrogen self-sufficiency. With the operating parameters now set, the operator determines the operating conditions of a multi-stage deoxygenation, isomerization and hydrogenation reactor within reactor system 104 and the operating conditions of the fractionation zone 107 to control the hydrocarbons being produced and separated so that the specifications and relative yields are met. Specific operating conditions will vary depending on the specific renewable feedstock and specifications of the desired products.

In the exemplary embodiment seen in FIG. 1, a renewable feed 101 is subjected to a pretreatment protocol 102 to reduce or remove contaminants. The resulting purified feed 103 is sent to the multi-stage reactor system 104 for deoxygenation, isomerization and hydrogenation. The multi-stage reactor system 104 contains at least one catalyst capable of catalyzing decarboxylation and/or hydrodeoxygenation of the purified feedstock 103 to remove oxygen.

Within the multi-stage reactor system 104, a deoxygenation effluent stream is directed to a hot high pressure hydrogen stripper, where gaseous components of the deoxygenation effluent are selectively stripped from liquid components. The separated gaseous components 105 are sent as to a hydrogen plant 111 where they serve as at least part of the feed and fuel of hydrogen plant 111. The liquid components of the deoxygenation effluent comprise primarily normal paraffins having a carbon number from about 8 to about 24 with a cetane number of about 60 to about 100.

Although not shown in FIG. 1, a portion of the liquid components may optionally form a recycle stream to be combined with the purified renewable feedstock stream 103 to create combined feed for the multi-stage reactor system 104. Also not shown in FIG. 1, another portion of recycle stream may be routed directly to the deoxygenation component of the multi-stage reactor system 104 and introduced at interstage locations to aid in temperature control. The remainder of liquid components is combined with hydrogen stream 112 and routed to an isomerization and hydrocracking reactor within the multi-stage reactor system 104.

The product of the isomerization and hydrocracking reactor containing a gaseous portion of hydrogen and propane and a branched-paraffin-enriched liquid portion may then be subjected to various processing steps, such as heat exchange and hydrogen separation, resulting in an effluent stream 106. Effluent stream 106 is then introduced into fractionation zone 107, where hydrocarbon by-product stream 110 containing naphtha, LPG, and other hydrocarbon by-products is separated as from a first product stream 108 containing hydrocarbons in the diesel fuel or additive range and a second product stream 109 containing hydrocarbons in the aviation fuel or additive range.

Although not shown in FIG. 1, the hydrocarbon by-product stream 110, or a portion separated therefrom, may be subjected to one or more amine absorbers, also called scrubbers, prior to being sent as feed and/or fuel for hydrogen plant 111. In embodiments utilizing an amine absorber, the amine chosen to be employed as an amine scrubber is capable of selectively removing at least both carbon dioxide and sulfur components such as hydrogen sulfide. Any suitable amine and operating conditions for an amine absorber may be employed. In some embodiments, a second amine scrubber may be used which contains an amine selective to hydrogen sulfide, but not selective to carbon dioxide. Again, any suitable amine and suitable operating conditions may be employed. The hydrocarbon by-product stream 110 is ultimately sent to hydrogen plant 111 for use as feed and fuel to generate hydrogen stream 112 in sufficient quantity that the system 100 is hydrogen self-sufficient.

Methods and systems for the generation of n-paraffins are similar to those described above for the generation of transportation fuels, with the exception that at least a portion of the n-paraffins generated during deoxygenation of the renewable feedstock is removed as a product stream without being subject to isomerization and hydrocracking. For instance, referring to FIG. 2, a renewable feed 201 may be subjected to a pretreatment protocol 202 as described above to remove or reduce contaminants in the renewable feed 201 and generate a purified feed 203. Purified feed 203 is sent to a deoxygenation reactor system 204 along with hydrogen stream 212 from hydrogen plant 211. The deoxygenation reactor system 204 contains at least one catalyst capable of catalyzing decarboxylation and/or hydrodeoxygenation of the purified feedstock 203 to remove oxygen.

In some embodiments, a deoxygenation effluent stream is directed to a hot high pressure hydrogen stripper within a deoxygenation reactor system 204, where gaseous components of the deoxygenation effluent are selectively stripped from liquid components. The separated gaseous components 205 are sent as to a hydrogen plant 211 where they serve as at least part of the feed and fuel of hydrogen plant 211. The liquid components of the deoxygenation effluent comprise primarily normal paraffins having a carbon number from about 8 to about 24 with a cetane number of about 60 to about 100.

From the deoxygenation reactor system 204, the liquid components are directed as effluent 206 to a fractionation zone 207, where by-product stream 210 containing naphtha, LPG, and other hydrocarbon by-products are separated from a first product stream 208 containing a heart cut of the desired n-paraffins and a second product stream 209 containing heavy paraffins that may be used as a cetane additive for various fuel products. In some embodiments, the desired n-paraffins include n-paraffins that are suitable for use as input materials for the production of detergents. Such n-paraffins are generally considered to be those with 10 to 13 carbons. As above, the by-product stream 210, or a portion separated therefrom, may be subjected to one or more amine absorbers prior to being sent as feed and/or fuel for hydrogen plant 211, where hydrogen stream 212 is generated in sufficient quantity that the system 200 is hydrogen self-sufficient.

The second product stream 209 is optionally sent to an isomerization and hydrocracking reactor system 213, where the second product stream 209 and hydrogen stream 214 are reacted to form a third product stream 216 containing hydrocarbons with a boiling point in the diesel fuel range. In these embodiments, a second by-product stream 215 containing naphtha, LPG, and other hydrocarbon by-products is also generated and sent to the hydrogen plant 211 for use as feed and/or fuel for generation of hydrogen streams 212 and 214.

As with the generation of transportation fuels, a user will set the conditions of the deoxygenation reactor system 204, fractionation zone 207, and optionally the isomerization and hydrocracking reactor system 213 such that sufficient quantities of separated gaseous components 205, by-product stream 210, and optionally second by-product stream 215 are generated to allow system 200 to be hydrogen self-sufficient. Specific operating conditions will vary depending on the specific renewable feed source and specifications of the desired products. In some embodiments, the amount of first and second by-product streams is about 5 wt % to about 40 wt %, such as about 10 wt % to about 40 wt %, such as about 15 wt % to about 40 wt %, such as about 20 wt % to about 40 wt %, of the fresh feed. In some embodiments, the amount of first and second by-product streams is about 10 wt % to about 30 wt % of the fresh feed.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for hydrogen self-sufficient production of hydrocarbons from a renewable feedstock, the method comprising:
providing a renewable feedstock;
contacting the renewable feedstock and hydrogen from a hydrogen stream with one or more catalysts to generate an effluent comprising n-paraffins and by-product hydrocarbons having 9 or fewer carbon atoms;
separating the by-product hydrocarbons from the effluent to generate a hydrocarbon by-product stream; and
feeding the hydrocarbon by-product stream to a hydrogen plant to generate the hydrogen stream;
wherein the by-product hydrocarbons constitute the entire feed and fuel of the hydrogen plant, and wherein no hydrogen is added from an external source.

2. The method of claim 1, wherein contacting the renewable feedstock and hydrogen from a hydrogen stream with one or more catalysts further comprises contacting the n-paraffins with a catalyst to generate an effluent comprising hydrocarbons with a boiling point in a diesel fuel boiling point range.

3. The method of claim 2, wherein the effluent generated by contacting the n-paraffins with the catalyst further comprises hydrocarbons with a boiling point in an aviation fuel boiling point range.

4. The method of claim 2, wherein the one or more catalysts comprise a hydrogenation and deoxygenation catalyst and an isomerization and hydrocracking catalyst.

5. The method of claim 2, wherein separating the by-product hydrocarbons from the effluent comprises fractionating the effluent into a first product stream comprising a diesel component with hydrocarbons with a boiling point in the diesel fuel boiling point range and a hydrocarbon by-product stream comprising by-product hydrocarbons having 9 or fewer carbon atoms.

6. The method of claim 5, wherein separating the by-product hydrocarbons from the effluent further comprises fractionating the effluent into a second product stream comprising an aviation component with hydrocarbons with a boiling point in the aviation fuel boiling point range.

7. The method of claim 1, wherein the amount of by-product stream produced is about 10 wt % to about 40 wt % of fresh feed.

8. The method of claim 1, wherein separating the by-product hydrocarbons having 9 or fewer carbon atoms from the effluent to generate a hydrocarbon by-product stream comprises fractionating the effluent into a first product stream comprising n-paraffins with 10 to 13 carbon atoms, a second product stream comprising hydrocarbons with 14 or more carbon atoms, and a first hydrocarbon by-product stream comprising by-product hydrocarbons having 9 or fewer carbon atoms.

9. The method of claim 8, further comprising subjecting the second product stream to an isomerization and hydrocracking catalyst in the presence of hydrogen to generate a second effluent comprising hydrocarbons with a boiling point in a diesel boiling point range and by-product hydrocarbons having 9 or fewer carbon atoms.

10. The method of claim 9, further comprising separating the by-product hydrocarbons from the second effluent to generate a third product stream comprising a diesel component with hydrocarbons with a boiling point in the diesel boiling point range and a second hydrocarbon by-product stream comprising by-product hydrocarbons having 9 or fewer carbon atoms.

11. The method of claim 10, further comprising using the second hydrocarbon by-product stream as feed or fuel for the hydrogen plant.

12. The method of claim 11, wherein the amount of first and second hydrocarbon by-product streams produced is about 10 wt % to about 40 wt % of fresh feed.

13. The method of claim 1, further comprising pre-treating the renewable feedstock under conditions suitable to at least reduce a portion of contaminants in the renewable feedstock prior to contact with a catalyst.

14. The method of claim 13, wherein the pre-treating the renewable feedstock comprises fractionating the renewable feedstock or contacting the renewable feedstock with an acidic ion exchange resin, an acid solution, or bleaching earth material.

15. The method of claim 1, wherein the renewable feedstock comprises at least one selected from the group consisting of glycerides, free fatty acids, fatty acid methyl esters, canola oil, corn oil, soy oils, rapeseed oil, soybean oil, colza oil, tall oil, sunflower oil, hempseed oil, olive oil, linseed oil, coconut oil, castor oil, peanut oil, palm kernel oil, mustard oil, cottonseed oil, tallow, yellow and brown greases, lard, train oil, fats in milk, fish oil, algal oil, sewage sludge, cuphea oil, camelina oil, jatropha oil, curcas oil, babassu oil, palm oil, fatty acid methyl esters, crambe oil, lard, kernel oil, used cooking oil, and animal fats.

16. The method of claim 15, wherein the renewable feedstock comprises one or more of palm oil, coconut oil, palm kernel oil, tallow, and lard.

17. A system for hydrogen self-sufficient production of hydrocarbons from a renewable feedstock, the system comprising:
a reaction zone configured to contain:
a hydrogenation and deoxygenation catalyst, wherein the reaction zone is configured to receive and contact a renewable feedstock and hydrogen gas with the hydrogenation and deoxygenation catalyst under reaction conditions effective to generate n-paraffins and hydrocarbon by-products having 9 or fewer carbon atoms; and
an isomerization and hydrocracking catalyst, wherein the reaction zone is configured to contact the n-paraffins from the hydrogenation and deoxygenation catalyst and hydrogen with the isomerization and hydrocracking catalyst under reaction conditions effective to generate an effluent comprising hydrocarbons with a boiling point in a diesel fuel boiling point range and hydrocarbon by-products having 9 or fewer carbon atoms;
a separation zone configured to receive an effluent from the reaction zone and fractionate the effluent into a first product stream comprising a diesel component with hydrocarbons with a boiling point in a diesel fuel boiling point range and a hydrocarbon by-product stream comprising by-product hydrocarbons having 9 or fewer carbon atoms; and
a hydrogen plant configured to receive the hydrocarbon by-product stream as feed and fuel for generation of hydrogen;
wherein the hydrogen plant is further configured such that by-product hydrocarbons constitute the entire feed and fuel of the hydrogen plant, and wherein no hydrogen is added to the system from an external source.

18. The system of claim 17, wherein the reaction zone is further configured such that the effluent generated by contacting the n-paraffins with the isomerization and hydrocracking catalyst further comprises hydrocarbons with a boiling point in an aviation fuel boiling point range, and the separation zone is further configured to separate the effluent into a second product stream comprising an aviation component with hydrocarbons in the aviation fuel boiling point range.

19. A system for hydrogen self-sufficient production of hydrocarbons from a renewable feedstock, the system comprising:
a first reaction zone configured to contain a hydrogenation and deoxygenation catalyst, wherein the first reaction zone is configured to receive and contact a renewable feedstock and hydrogen gas with the hydrogenation and deoxygenation catalyst under reaction conditions effective to generate n-paraffins and hydrocarbon by-products having 9 or fewer carbon atoms;
a first separation zone configured to receive an effluent from the first reaction zone and fractionate the effluent into a first product stream comprising n-paraffins with 10 to 13 carbon atoms, a second product stream comprising hydrocarbons with 14 or more carbon atoms, and a first hydrocarbon by-product stream comprising by-product hydrocarbons having 9 or fewer carbon atoms; and
a hydrogen plant configured to receive the first hydrocarbon by-product stream as feed and fuel for generation of hydrogen;
wherein the hydrogen plant is further configured such that by-product hydrocarbons constitute the entire feed and fuel of the hydrogen plant, and wherein no hydrogen is added to the system from an external source.

20. The system of claim 19, further comprising:
a second reaction zone configured to contain an isomerization and hydrocracking catalyst, wherein the second reaction zone is configured to receive and contact the second product stream and hydrogen gas with the isomerization and hydrocracking catalyst under reaction conditions effective to generate an effluent comprising hydrocarbons in the diesel boiling point range and by-product hydrocarbons having 9 or fewer carbon atoms; and
a second separation zone configured to receive an effluent from the second reaction zone and fractionate the effluent into a third product stream comprising a diesel component with hydrocarbons in a diesel boiling point range and a second hydrocarbon by-product stream comprising by-product hydrocarbons having 9 or fewer carbon atoms;
wherein the hydrogen plant is further configured to receive the second hydrocarbon by-product stream as feed and fuel for generation of hydrogen.
